(19) United States
(12) Patent Application Publication
(10) Pub. No.: US 2026/0007395 A1
(43) Pub. Date: Jan. 8, 2026

(54) Catheter-Image Guided Biopsy Needle with Needle Tip Having Multiple Degrees Of Freedom

(71) Applicant: Leadoptik Inc., San Jose, CA (US)

(72) Inventors: Christopher Khiemvinh Huynh, San Jose, CA (US); Mohammadreza Khorasaninejad, San Jose, CA (US)

(21) Appl. No.: 19/259,860

(22) Filed: Jul. 3, 2025

Related U.S. Application Data
(60) Provisional application No. 63/667,616, filed on Jul. 3, 2024.

(51) Int. Cl. A61B 10/04

(52) U.S. Cl. CPC A61B 10/04; A61B 2010/045

(57) ABSTRACT

A catheter-based biopsy system integrates real-time optical coherence tomography (OCT) imaging with a multi-degree-of-freedom biopsy needle for precise tissue sampling within luminal organs. The system comprises a segmented handle, an outer sheath, a steerable needle, and an imaging stylet housed within the needle. The imaging stylet includes a metasurface-based optical lens for acquiring cross-sectional images through an imaging window in the needle. Independent handle segments control the extension of the needle and the positioning of the imaging stylet. The system supports multiple operational modes, including imaging, image-guided biopsy, and tissue retrieval, with optional encoder-based tracking for enhanced navigation. The integrated design enables targeted fine needle aspiration and biopsy under direct visualization, improving diagnostic accuracy and procedural efficiency.

### RELATED APPLICATION

The present Application claims the priority of U.S. Provisional Application No. 63/667,616, filed Jul. 3, 2024, titled “Catheter-Image Guided Biopsy Needle with Needle Tip Having Multiple Degrees of Freedom”, the disclosure of which is incorporated by reference herein in its entirety.

### TECHNICAL FIELD

The present disclosure relates to the field of surgical implements, and more specifically to an imaging and biopsy device having multiple degrees of freedom.

### BACKGROUND

Accurate diagnosis and treatment of diseases in internal organs such as the pulmonary airways, the coronary arteries, and the gastrointestinal tract are difficult due to the inaccessibility of lesions. This is the main drive behind the miniaturization of optical imaging, illumination (for therapeutic purposes), and navigation (guiding the biopsy needle, targeted drug delivery tools/needles, or surgical devices) systems. Miniaturization often comes at the cost of reducing functionality. For example, to achieve a smaller form factor imaging system, it is difficult to cascade several components or have electrical or mechanical actuators. These limit the functionality of imaging systems such as depth of field that determine signal-to-noise ratio in imaging applications.

### BRIEF DESCRIPTION OF THE DRAWINGS

The disclosure will be understood more fully from the detailed description given below and from the accompanying figures of embodiments of the disclosure. The figures are used to provide knowledge and understanding of embodiments of the disclosure and do not limit the scope of the disclosure to these specific embodiments. Furthermore, the figures are not necessarily drawn to scale.

FIG. 1 illustrates a biopsy device, in accordance with an example embodiment.

FIGS. 2A and 2B illustrate a distal end of the biopsy device of FIG. 1 during various modes of operation, in accordance with an example embodiment.

FIG. 3 illustrates a needle of the biopsy device of FIG. 1, in accordance with an example embodiment.

FIG. 4 illustrates an imaging assembly for an imaging stylet of the biopsy device of FIG. 1, in accordance with an example embodiment.

FIG. 5 illustrates an optically transmissive housing for the imaging stylet of the biopsy device of FIG. 1, in accordance with an example embodiment.

FIG. 6 illustrates an outer sheath of the biopsy device of FIG. 1, in accordance with an example embodiment.

FIG. 7 illustrates a segmented handle including a first handle piece, a second handle piece, and a third handle piece of the biopsy device of FIG. 1, in accordance with an example embodiment.

FIG. 8 illustrates a proximal end of the biopsy device of FIG. 1, including a driving unit and optical connector to an imaging console (not shown), in accordance with an example embodiment.

FIG. 9 illustrates a steering assembly which may be used with the biopsy device of FIG. 1, in accordance with an example embodiment.

FIG. 10 illustrates the biopsy device of FIG. 1 coupled to the steering assembly of FIG. 9, in accordance with an example embodiment.

FIG. 11 illustrates a fourth handle piece of the steering assembly of FIG. 9, in accordance with an example embodiment.

FIGS. 12A and 12B illustrate an articulating sheath of the steering assembly of FIG. 9, in accordance with an example embodiment.

FIG. 13 illustrates a biopsy device, in accordance with an example embodiment.

FIG. 14 illustrates the segmented handle of FIG. 7 configured for a home operating mode of the biopsy device, in accordance with an example embodiment.

FIG. 15 illustrates the distal end of FIGS. 2A and 2B configured for the home operating mode of the biopsy device, in accordance with an example embodiment.

FIG. 16 illustrates the segmented handle of FIG. 7 configured for an imaging operating mode of the biopsy device, in accordance with an example embodiment.

FIG. 17 illustrates the distal end of FIGS. 2A and 2B configured for the imaging operating mode of the biopsy device, in accordance with the embodiment of FIG. 16.

FIG. 18 illustrates the segmented handle of FIG. 7 configured for an image-guided biopsy operating mode of the biopsy device, in accordance with an example embodiment.

FIG. 19 illustrates the distal end of FIGS. 2A and 2B configured for the image-guided biopsy operating mode of the biopsy device, in accordance with the embodiment of FIG. 18.

FIG. 20 illustrates the segmented handle of FIG. 7 configured for a biopsy operating mode of the biopsy device, in accordance with an example embodiment.

FIG. 21 illustrates the distal end of FIGS. 2A and 2B configured for the biopsy operating mode of the biopsy device, in accordance with an the embodiment of FIG. 20.

FIG. 22 illustrates the segmented handle of FIG. 7 configured for an additional operating mode of the biopsy device, in accordance with an example embodiment.

FIG. 23 illustrates the distal end of FIGS. 2A and 2B configured for the additional operating mode of the biopsy device, in accordance with an the embodiment of FIG. 22.

FIG. 24 illustrates the segmented handle of FIG. 7 configured for a retrieval operating mode of the biopsy device, in accordance with an example embodiment.

FIG. 25 illustrates the distal end of FIGS. 2A and 2B configured for the retrieval operating mode of the biopsy device, in accordance with the embodiment of FIG. 24.

FIGS. 26A and 26B illustrate a strain relief module of the biopsy device of FIG. 1 in accordance with various embodiments.

FIGS. 27A and 27B illustrate the driving unit of FIG. 8 in accordance with various embodiments.

FIGS. 28A and 28B illustrate a biopsy device in accordance with various embodiments.

FIG. 29 depicts a diagram of an example computer system in which embodiments of the present disclosure may operate.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed. Embodiments of the disclosure are described more fully hereinafter with reference to the accompanying drawings; however, alternative configurations and embodiments are also possible without departing from the scope of the present disclosure. Thus, the present disclosure should not be construed as limited to the embodiments expressly set forth herein. Rather, the illustrated and described embodiments are provided as examples to convey the scope of the disclosure to those skilled in the art. In the drawings, the size and relative sizes of layers and regions may be exaggerated for clarity.

### Configuration Overview

Aspects of the present disclosure relate to a catheter-image guided biopsy needle with a needle tip having multiple degrees of freedom, particularly a catheter-integrated, image-guided biopsy needle system configured for real-time optical coherence tomography (OCT) imaging or Raman Spectroscopy (or other imaging and spectroscopy modalities) and tissue sampling within luminal organs such as the tracheobronchial tree. The system includes a biopsy needle with a needle tip capable of multiple degrees of freedom (DoF), enabling precise localization and navigation and tissue acquisition in anatomically complex or difficult-to-access regions.

The biopsy device includes a modular assembly including a handle assembly, an outer sheath, a biopsy needle, and an imaging stylet (also referred to as an imaging assembly). The outer sheath is may be a clear Nylon extrusion tube with an embedded radiopaque (RO) marker band of platinum-iridium alloy (e.g., 90% Pt/10% Ir) to facilitate fluoroscopic visualization. The outer sheath may be dimensioned for compatibility with endoscopes having a working channel diameter of at least 1.7 mm, and may provide an insertable length of approximately 110 cm.

The handle assembly incorporates independent actuators for the needle and the imaging stylet in the form of three or more adjustable handle pieces configured for independent translational and rotational motion. A needle actuator handle piece (e.g., a second handle piece) enables controlled extension and retraction of the biopsy needle from the distal end of the outer sheath, and can include demarcations allowing for precise depth control of the needle (e.g., up to 4 centimeters). An imaging stylet actuator handle piece (e.g., a third handle piece) similarly allows for controlled positioning of the imaging stylet relative to the needle tip, including alignment with an imaging window (e.g., a discontinuity) located at the distal end of the needle.

In various embodiments, the biopsy needle is a 21-gauge stainless steel shaft with a beveled tip for tissue penetration. The proximal end of the needle can include a helical laser-cut pattern encapsulated within a Nylon or Pebax extrusion, enabling the needle to conform to tight anatomical curvatures with a bending radius of up to 210°. The imaging window is integrated into the needle wall to permit optical access to the surrounding tissue by the imaging stylet.

The imaging stylet may be a flexible, transparent Nylon tube housing a metasurface-based optical lens system and associated optical components. These components are configured to focus and collect backscattered light from biological tissue, enabling real-time OCT imaging. A proximal end of the imaging stylet terminates in a fiber optic connector that interfaces with an external imaging console (e.g., a LIA Console), which processes and displays cross-sectional images of tissue microstructure.

The biopsy device is configurable between a plurality of operating modes, each mode corresponding to a specific configuration of the needle and imaging stylet. In a first operating mode (a home mode), both the needle and imaging stylet are retracted within the outer sheath to facilitate safe insertion through a bronchoscope (a delivery device for the biopsy device). In a second operating mode (an imaging mode), the imaging stylet is advanced to enable 360° OCT imaging for lesion localization or navigation. In a third operating mode (an image-guided biopsy mode), the needle is deployed to the target site under real-time imaging guidance. In a fourth operating mode (a biopsy mode), the imaging stylet is retracted, and a preloaded aspiration syringe may be connected (e.g., via a Luer connector) to collect tissue samples. In a fifth operating mode (a retrieval mode), the catheter is withdrawn, and the sample is ejected by extending the needle and advancing the imaging stylet.

In some embodiments, a strain relief adapter with an integrated encoder may be used to dock the catheter to the bronchoscope and track axial movement of the catheter. Positional data from the encoder can be correlated with imaging data of the biopsy device to enhance navigation and targeting accuracy. In certain embodiments, the catheter may be used in conjunction with a steering assembly, either integrated or separate from the handle assembly, to provide additional articulation (e.g., up to) 180° for accessing peripheral lesions.

The biopsy device may be used to perform fine needle aspiration (FNA) and fine needle biopsy (FNB) under real-time OCT visualization, enabling precise targeting and sampling of endobronchial lesions, peripheral lung nodules, and other intraluminal abnormalities. Integration of imaging and biopsy functionality within a single catheter platform can reduce device exchanges, minimize procedural time, and enhance diagnostic yield.

In some aspects, the techniques described herein relate to a device to facilitate imaging within a human body, including: a handle positioned toward a proximal end of the device, the handle including a first handle piece, a second handle piece, and a third handle piece, the handle pieces being adjustable between a plurality of states to control a plurality of operating modes of the device based on translational and rotational motion of the first handle piece, the second handle piece, and the third handle piece; a sheath within the device, the sheath operably connected to a first handle piece such that the first handle piece controls the translational motion of the sheath within the device; a needle within the sheath, the needle including a needle tip of the needle positioned toward a distal end of the device, and the needle being operably connected to the second handle piece such that the second handle piece controls translational and rotational motion of the needle within the sheath; and an imaging stylet positioned within the needle, the imaging stylet including an imaging assembly and an optically transmissive housing for imaging within the human body, and the imaging stylet being operably connected to the third handle piece such that the third handle piece controls translational and rotational motion of the imaging stylet within the sheath.

In some aspects, the techniques described herein relate to a device, further including a fourth handle piece configured for rotational motion and operably connected to a steerable articulating segment of the sheath, the steerable articulating segment configured to guide the needle to a target within the human body.

In some aspects, the techniques described herein relate to a device, further including a driving unit positioned at a proximal end of the device, the driving unit including: an optically transmissive fiber for rotating the imaging assembly perform 360-degree imaging; and an optical connector having a textured pattern formed in a wall of the optical connector, the pattern encoding a unique identifier of the device.

In some aspects, the techniques described herein relate to a device wherein, in a home operating mode, the third handle piece is translationally moved apart from the second handle piece toward the proximal end of the device, causing the imaging stylet to be positioned within the needle tip, and the second handle piece is translationally moved apart from the first handle piece toward the proximal end of the device, causing the needle tip to be positioned within the sheath.

In some aspects, the techniques described herein relate to a device wherein, in an imaging operating mode, the third handle piece is translationally moved toward the distal end of the device such that the third handle piece abuts the second handle piece, causing the imaging stylet to project outward from the needle and the sheath for imaging.

In some aspects, the techniques described herein relate to a device wherein, in an image-guided biopsy operating mode, the second handle piece is translationally moved toward the distal end of the device such that the second handle piece abuts the first handle piece, causing the needle to project outward from the sheath, and the third handle piece remains stationary toward the proximal end of the device, aligning a lens of the imaging assembly with a discontinuity in the needle tip.

In some aspects, the techniques described herein relate to a device wherein, in a biopsy operating mode, the third handle piece is moved to a maximum position of its range of translational movement toward the proximal end of the device, causing the imaging stylet to vacate the needle tip and be positioned within the sheath, and the second handle piece is translationally moved in a reciprocating motion to agitate the needle and collect a tissue sample within the needle tip.

In some aspects, the techniques described herein relate to a device wherein, in an ejection operating mode, the third handle piece is translationally moved toward the distal end of the device such that the third handle piece abuts the second handle piece, causing the imaging stylet to eject a tissue sample from the needle tip.

In some aspects, the techniques described herein relate to a device, wherein the imaging stylet is capable of three degrees of translational motion and two degrees of rotational motion.

In some aspects, the techniques described herein relate to a method including: adjusting a handle including a first handle piece, a second handle piece, and a third handle piece between a plurality of states to control a plurality of operating modes of a biopsy device based on translational and rotational motion of the first handle piece, the second handle piece, and the third handle piece; wherein a sheath within the device is operably connected to the first handle piece such that the first handle piece controls translational motion of the sheath within the device; wherein a needle within the sheath is operably connected to the second handle piece such that the second handle piece controls translational and rotational motion of the needle within the sheath, the needle including a needle tip; and wherein an imaging stylet within the needle is operably connected to the third handle piece such that the third handle piece controls translational and rotational motion of the imaging stylet, the imaging stylet including an imaging assembly and an optically transmissive housing for imaging within a human body.

In some aspects, the techniques described herein relate to a method, further including adjusting a fourth handle piece of the device configured for rotational motion and operably connected to a steerable articulating segment of the sheath, the steerable articulating segment configured to guide the needle to a target within the human body.

In some aspects, the techniques described herein relate to a method, further including rotating, by an optically transmissive fiber, the imaging assembly to perform 360-degree optical coherence tomography (OCT) imaging.

In some aspects, the techniques described herein relate to a method, further including selecting a home operating mode for the device by translationally moving the third handle piece apart from the second handle piece toward a proximal end of the device, causing the imaging stylet to be positioned within the needle tip, and translationally moving the second handle piece apart from the first handle piece toward a proximal end of the device, causing the needle tip to be positioned within the sheath.

In some aspects, the techniques described herein relate to a method, further including selecting an imaging operating mode for the device by translationally moving the third handle piece toward a distal end of the device such that the third handle piece abuts the second handle piece, causing the imaging stylet to project outward from the needle and the sheath for imaging.

In some aspects, the techniques described herein relate to a method, further including selecting an image-guided biopsy operating mode for the device by translationally moving the second handle piece toward a distal end of the device such that the second handle piece abuts the first handle piece, causing the needle to project outward from the sheath, and maintaining a position of the third handle piece toward a proximal end of the device, aligning a lens of the imaging assembly with a discontinuity in the needle tip.

In some aspects, the techniques described herein relate to a method, further including selecting a biopsy operating mode for the device by translationally moving the third handle piece to a maximum position of its range of translational movement toward a proximal end of the device, causing the imaging stylet to vacate the needle tip and be positioned within the sheath, and translationally moving the second handle piece in a reciprocating motion to agitate the needle and collect a tissue sample within the needle tip.

In some aspects, the techniques described herein relate to a method, further including selecting an ejection operating mode for the device by translationally moving the third handle piece toward a distal end of the device such that the third handle piece abuts the second handle piece, causing the imaging stylet to eject a tissue sample from the needle tip.

In some aspects, the techniques described herein relate to a method, further including moving the imaging stylet along three axes of translational motion and two axes of rotational motion.

In some aspects, the techniques described herein relate to a method, further including adjusting an angle of curvature of the sheath by translational or rotational movement of a control arm of a steering assembly of the device.

In some aspects, the techniques described herein relate to a method, further including tracking movement of the device within the human body by accessing movement data generated by an encoder and imaging data generated by the imaging stylet.

### Example Biopsy Devices

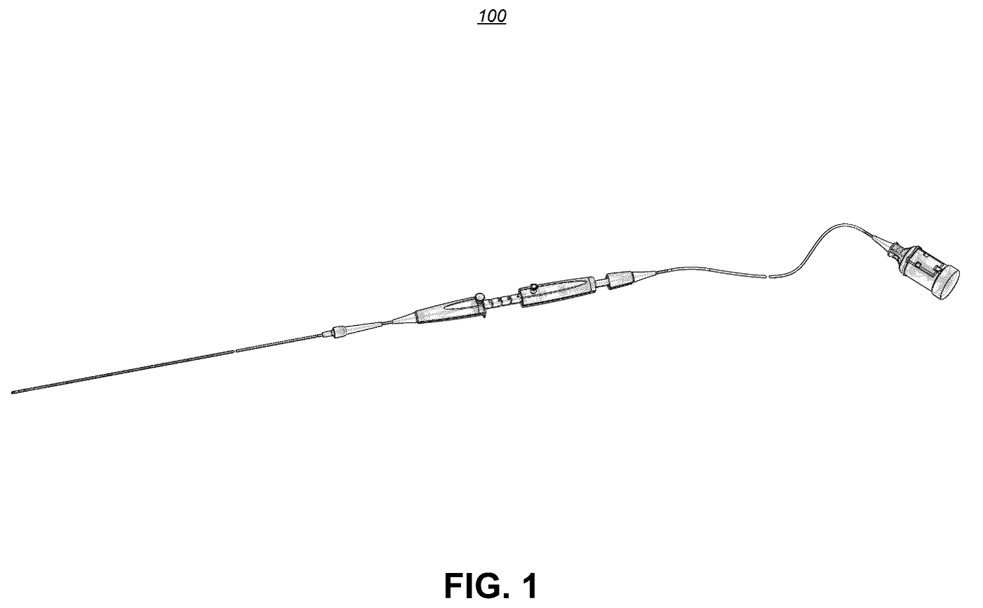

Referring initially to FIGS. 1-8, a biopsy device 100 (FIG. 1) is shown in accordance with an example embodiment. The biopsy device 100 includes a outer sheath 600 (FIG. 6) within the device 100, the outer sheath 600 operably connected to a first handle piece 710 (FIG. 7) such that the first handle piece 710 controls translational motion of the outer sheath 600 within the device 100. The outer sheath 600 is may be a clear Nylon extrusion tube with an embedded radiopaque (RO) marker band of platinum-iridium alloy (e.g., 90% Pt/10% Ir) to facilitate fluoroscopic visualization, but other sheath materials are also possible. The outer sheath 600 may be dimensioned for compatibility with endoscopes having a working channel diameter of at least, e.g., 1.7 mm, and may provide an insertable length of, e.g., 110 cm.

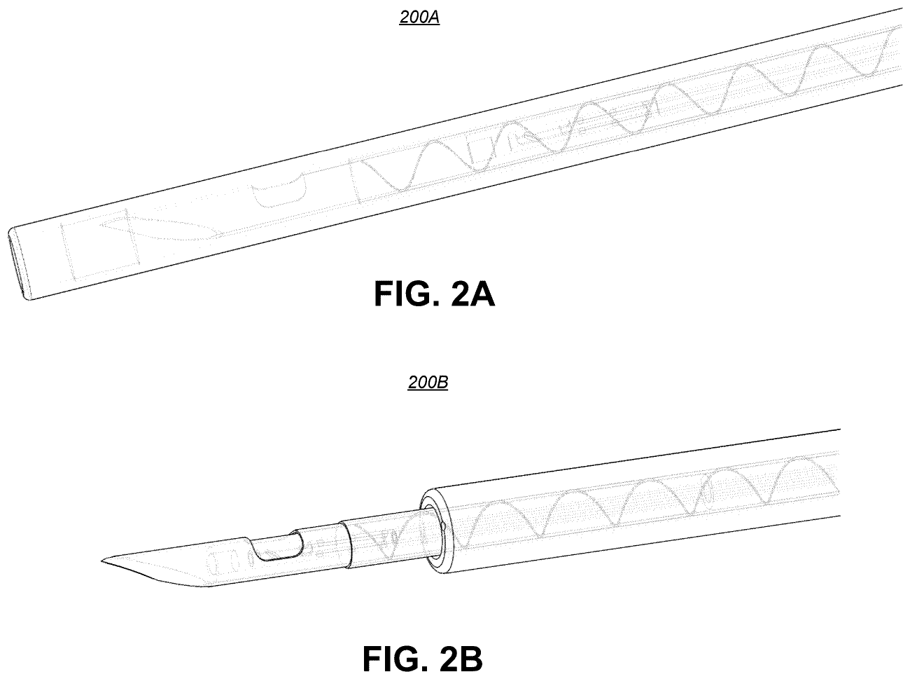

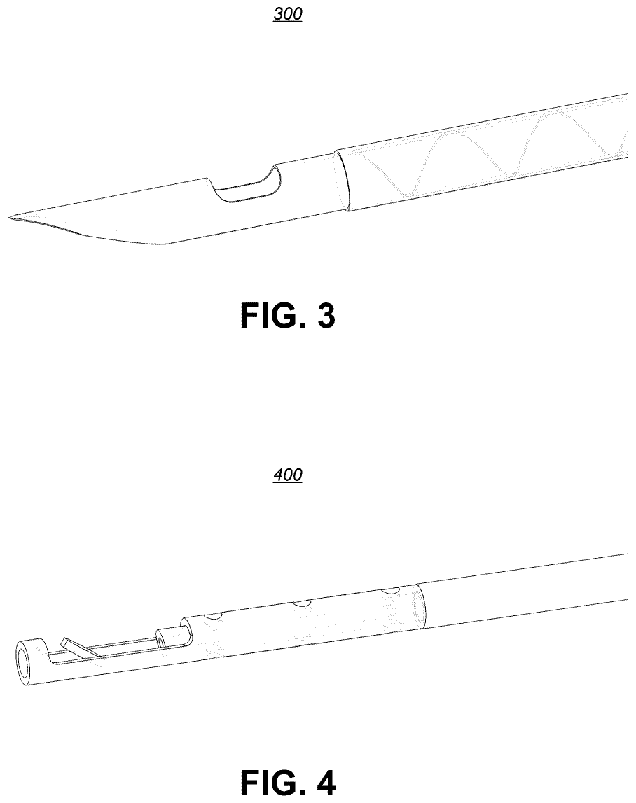

The biopsy device 100 includes a needle 300 (FIG. 3) within the outer sheath 600, the needle 300 includes a needle tip positioned toward a distal end of the device 100. The needle 300 is operably connected to a second handle piece 720 (FIG. 7) such that the second handle piece 720 controls translational and rotational motion of the needle 300 within the outer sheath 600. For example, FIG. 2A illustrates a retracted state of the needle 300 within the outer sheath 600, and FIG. 2B illustrates an extended (e.g., projecting) state of the needle 300 from a distal end of the outer sheath 600. In some embodiments, the needle 300 is a 21-gauge stainless steel shaft with a beveled tip for tissue penetration, but other needles are also possible. The proximal end of the needle 300 can include a helical laser-cut pattern encapsulated within a Nylon extrusion, enabling the needle 300 to conform to tight anatomical curvatures with a bending radius of up to, e.g., 210° (e.g., using actuation of one or more handle pieces). An imaging window (e.g., a discontinuity in the needle wall) is integrated into the needle wall to permit optical access to the surrounding tissue by an imaging stylet.

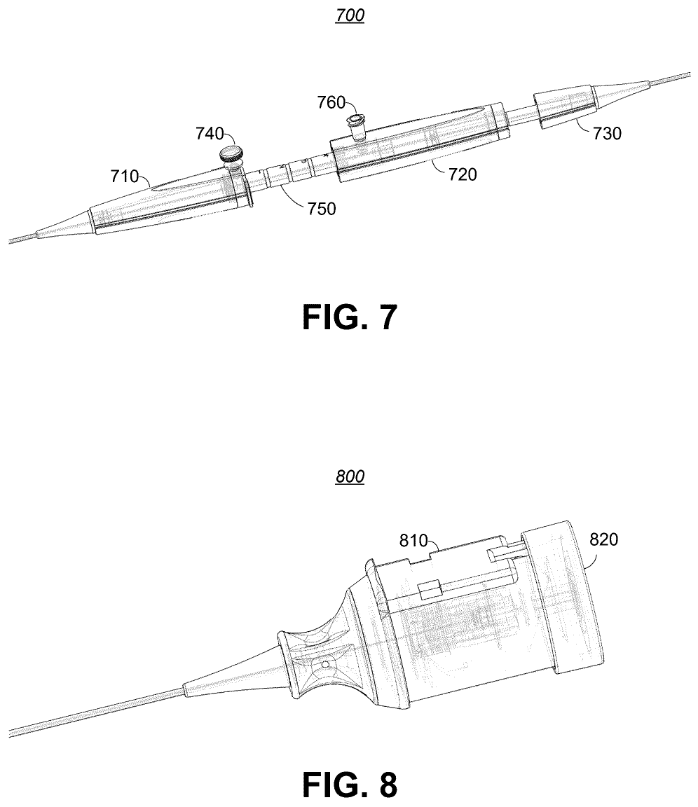

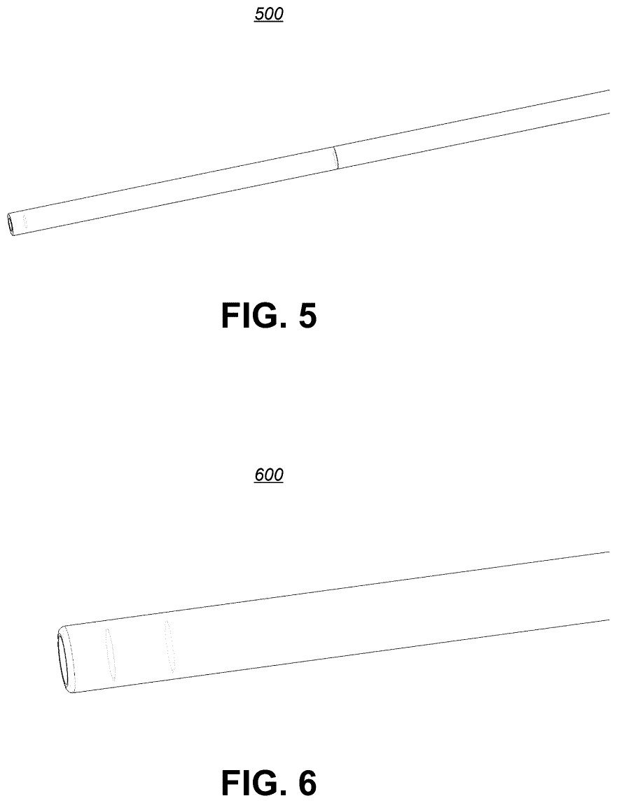

The biopsy device 100 includes an imaging stylet 400 including an imaging assembly (FIG. 4) and an optically transmissive housing (FIG. 5) for performing imaging within a human body. The imaging stylet 400 is operably connected to a third handle piece 730 (FIG. 7) such that the third handle piece 730 controls translational and rotational motion of the imaging stylet 400 within the outer sheath 600. The imaging stylet 400 may be a flexible, transparent Nylon tube housing a metasurface-based optical lens system and associated optical components. These components are configured to focus and collect backscattered light from biological tissue, enabling real-time OCT imaging. The housing may comprise a combination of two or three distinct tubular components, wherein the tubular components may include transparent tubing, braided tubing, or other non-transparent tubing. A proximal end of the imaging stylet 400 terminates in a fiber optic connector that interfaces with an external imaging console (e.g., a LIA Console), which processes and displays cross-sectional images of tissue microstructure. Various forms of imaging stylet are possible and may not all be illustrated here.

The first handle piece 710, second handle piece 720, and third handle piece 730 collectively form a segmented handle 700 (also referred to as a handle assembly), the handle pieces being adjustable between a plurality of states to control a plurality of operating modes of the biopsy device 100 based on their translational and/or rotational motion. In some embodiments, the first handle piece 710 includes a locking mechanism 740 (e.g., a thumb screw) configured to limit translational and rotational motion of the second handle piece 720. This has the effect of limiting translational and rotational motion of the needle 300 relative to the outer sheath 600. Demarcations 750 on the second handle piece 720 allow for precise depth control of the needle 300 (for example, up to and including 4 centimeters of extension beyond the distal end of the outer sheath 600). The second handle piece 720 includes a port 760 (e.g., a Luer connector) for connecting an aspiration syringe, such as to apply a negative pressure within the outer sheath 600.

A driving unit 800 (FIG. 8) is provided at the proximal end of the biopsy device 100 for connecting the fiber optic connector of the imaging stylet 400 to the external imaging console. The driving unit 800 functions as a keyed optical connector 820 for connecting to the external imaging console, and includes a textured pattern formed in a side wall 810 which encodes a unique identifier of the biopsy device 100. For example, the unique identifier may include a serial number of the biopsy device 100 or an indication of a current operating mode of the biopsy device 100. The unique identifier may also trigger a specific version of application software or cause a user interface to be displayed on the imaging console upon connection to the imaging console. In the illustrated embodiment, the unique identifier is represented as a plurality of indentations in the side wall 810 configured to be read by the external imaging console. In various embodiments, the size, depth, and location of one or more projections or indentations in the side wall 810 may be used to represent the unique identifier. To provide further example, the side wall 810 may be slotted like a “key” such that, when inserted into a receiver, the side wall 810 indentations indicate functionality of the attached device. However, any implementation of the unique identifier known to those skilled in the art may be used.

The driving unit 800 can interface with a motor of a connected imaging console, the motor configured to rotate the imaging stylet 400 by way of an optically transmissive fiber which terminates in the driving unit 800. In some embodiments, the motor is configured to rotate the imaging assembly to perform 360-degree optical coherence tomography (OCT) imaging or another type of imaging.

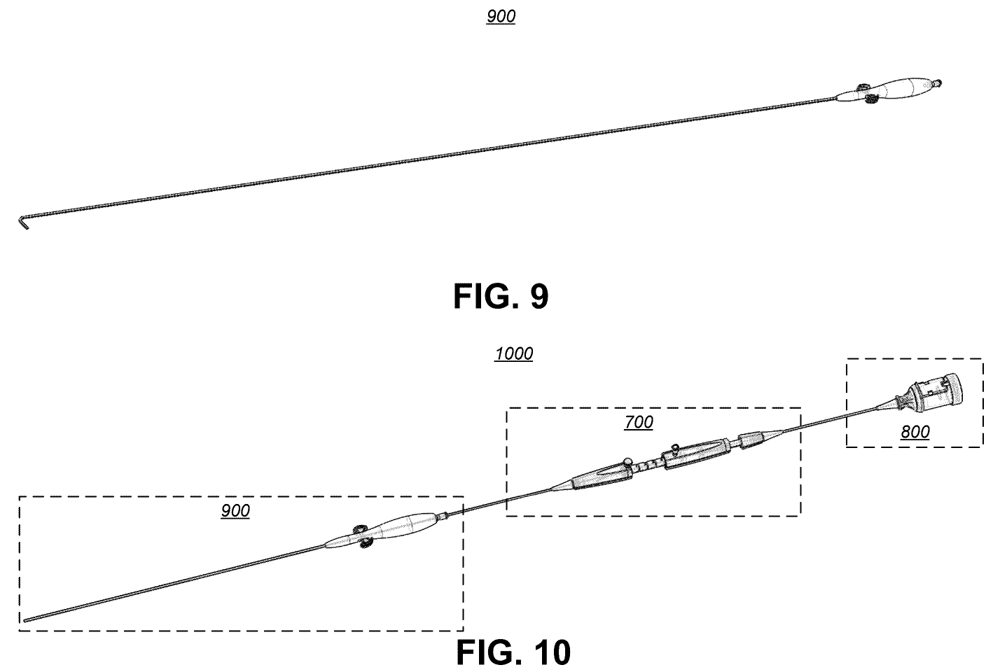

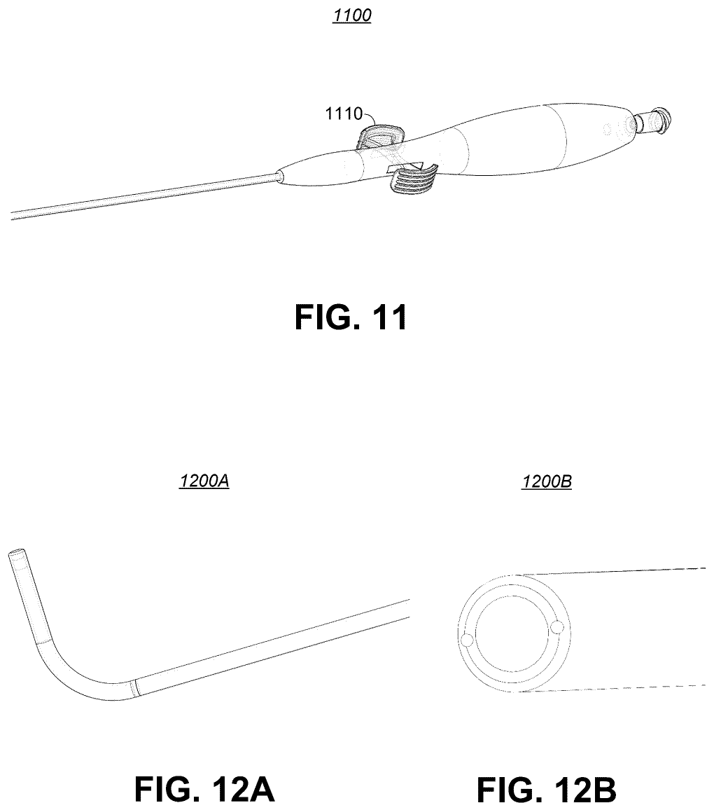

FIGS. 9-13 illustrate a steering assembly 900 (FIG. 9) of the biopsy device 100 of FIG. 1. In various embodiments, the steering assembly 900 offers additional degrees of freedom for guiding the needle 300 and imaging stylet 400 within a human body. The steering assembly 900 includes a fourth handle piece 1100 (FIG. 11) including a control arm 1110 which is operably connected to a steerable articulating segment 1200A (FIG. 12A) of the outer sheath 600. The steerable articulating segment 1200A is illustrated with a 90° angle of curvature by way of example only. By selectively rotating the control arm 1110, a user can modify the angle of curvature of the steerable articulating segment 1200A. In an embodiment, the angle of curvature may be in a range from 0° (where the outer sheath 600 is completely straight) through 180° (where the distal end of the outer sheath 600 is curved back toward the proximal end of the biopsy device 100) to access peripheral targets with the human body. In various embodiments, the steerable articulating segment 1200A may be manipulated by translational or rotational movement of the control arm 1110 (and/or movements of handle pieces).

In some embodiments, the distal end of the outer sheath 600 includes an illumination output 1200B (FIG. 12B) configured to illuminate the environment during imaging or biopsy. The illumination output 1200B can include, e.g., one or more optical fibers embedded in the outer sheath 600 configured to transmit light from the driving unit 800 (or a connected imaging console) into the human body. The distal end of the outer sheath 600 may further include the radiopaque marker band to observe the position of the outer sheath 600 (and by extension, the needle 300 and imaging stylet 400) within the human body.

In one embodiment, the steering assembly 900 may an extension of outer sheath 600, the steering assembly 900 configured to receive the needle 300 and imaging stylet 400 of the biopsy device 100 via a port (e.g., a Luer connector) at a proximal end of the fourth handle piece 1100. This configuration is illustrated by FIG. 10, showing the segmented handle 700, driving unit 800, and steering assembly 900 connected by portions of the outer sheath 600. Advantageously, this modular configuration allows for the steering assembly 900 to be connected or disconnected from the biopsy device 100 based on whether the added degree of articulation offered by the steerable articulating segment 1200A is required to reach a target. The steering assembly 900 offers additional degrees of motion to the imaging stylet 400, including three axes of translational motion and two axes of rotational motion.

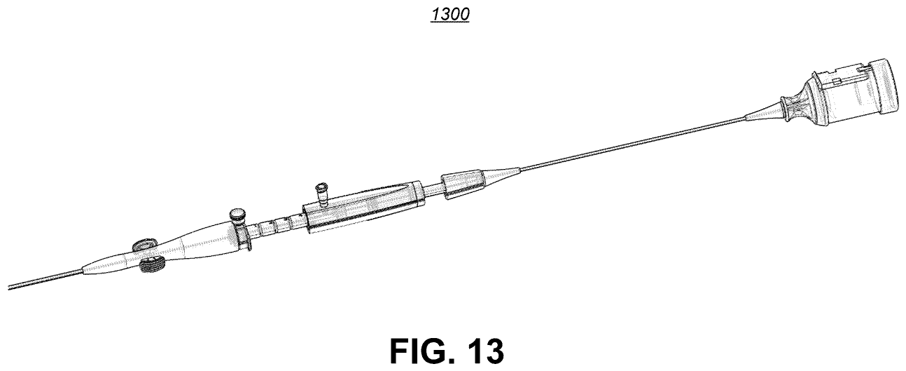

In another embodiment, the fourth handle piece 1100 of the steering assembly 900 is integrated within a handle piece of a biopsy device, offering a higher degree of articulation in a compact device. For example, the biopsy device 1300 of FIG. 13 integrates the fourth handle piece 1100 of FIG. 11 into the first handle piece 710 of the segmented handle 700 of FIG. 7. This can offer ergonomic advantages for the operator and improve ease of use by moving the control arm 1110 of the steering assembly 900 closer to the segmented handle 700 as compared to the embodiment of FIG. 10. In various embodiments, the fourth handle piece 1100 may be integrated into any handle piece of the segmented handle 700, including the second handle piece 720 or the third handle piece 730. However, various implementations of the steering assembly 900 integrated within a biopsy device are possible.

### Example Operating Modes

The biopsy devices described herein can operate in a plurality of user-selectable operating modes. To select an operating mode, a user modifies a physical configuration of the biopsy device to perform one or more functions associated with that operating mode, such as imaging of the human body, tissue sample biopsy, tissue sample retrieval, and the like. For ease of discussion, the following description will refer to the plurality of operating modes in the context of the biopsy device 100 of FIG. 1. However, it will be understood that the plurality of operating modes is applicable to any embodiment of the biopsy device disclosed herein.

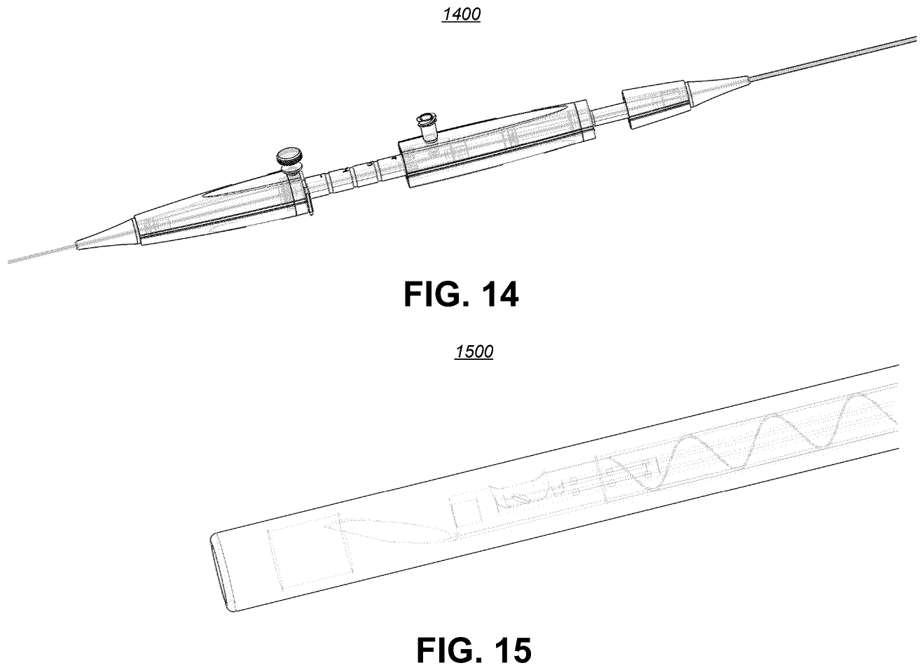

In a home operating mode (e.g., a home position), the biopsy device 100 is configured for storage and insertion into the human body. FIG. 14 illustrates the segmented handle 700 of the biopsy device 100 in the home operating mode. FIG. 15 illustrates the distal end of the outer sheath 600, including the needle 300 and imaging stylet 400 retracted within the outer sheath 600. To select the home operating mode, the user translationally moves (e.g., by sliding) the third handle piece 730 apart from the second handle piece 720 and translationally moves the second handle piece 720 apart from the first handle piece 710. These translational movements cause the imaging stylet 400 to be positioned within (e.g., retracted inside) the needle 300, and the needle 300 to be positioned within the distal end of the outer sheath 600, respectively. The home operating mode may be used to, for example, feed the outer sheath 600 through an insertion port and toward a working channel of a bronchoscope.

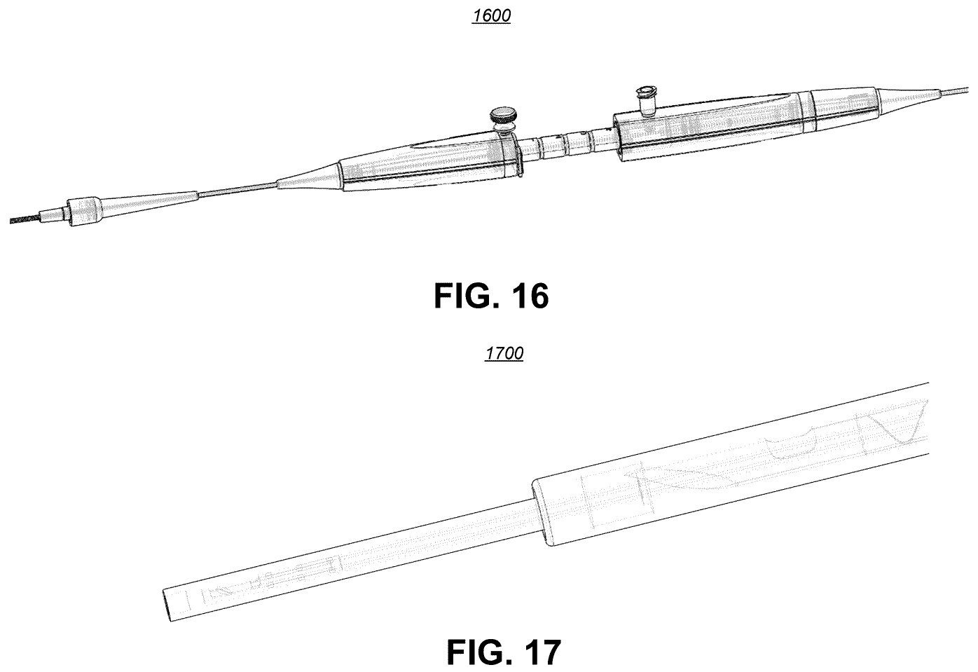

In an imaging operating mode, the biopsy device 100 is configured to perform imaging, such as optical coherence tomography (OCT), via the imaging stylet 400. FIG. 16 illustrates the segmented handle 700 of the biopsy device 100 in the imaging operating mode. FIG. 17 illustrates the distal end of the outer sheath 600, including the imaging stylet 400 projecting outward from the needle 300 and the outer sheath 600. The imaging operating mode may be used to identify the location of, e.g., a nodule within the human body before a biopsy is performed. To select the imaging operating mode (provided that the biopsy device 100 is initially in the home operating mode), the user translationally moves the third handle piece 730 toward the distal end of the biopsy device 100 such that the third handle piece 730 abuts the second handle piece 720, causing the imaging stylet 400 to project outward from the needle 300 and the outer sheath 600 for imaging. Notably, the second handle piece 720 remains stationary at its position in the home operating mode (and may be fixed in place by the locking mechanism 740), causing the needle 300 to remain fully recessed (e.g., retracted) within the outer sheath 600. During imaging, the motor of the imaging console which is connected to the imaging stylet 400 rotates the imaging stylet 400 via the fiber optic connector to perform imaging, such as 360° OCT imaging for lesion localization.

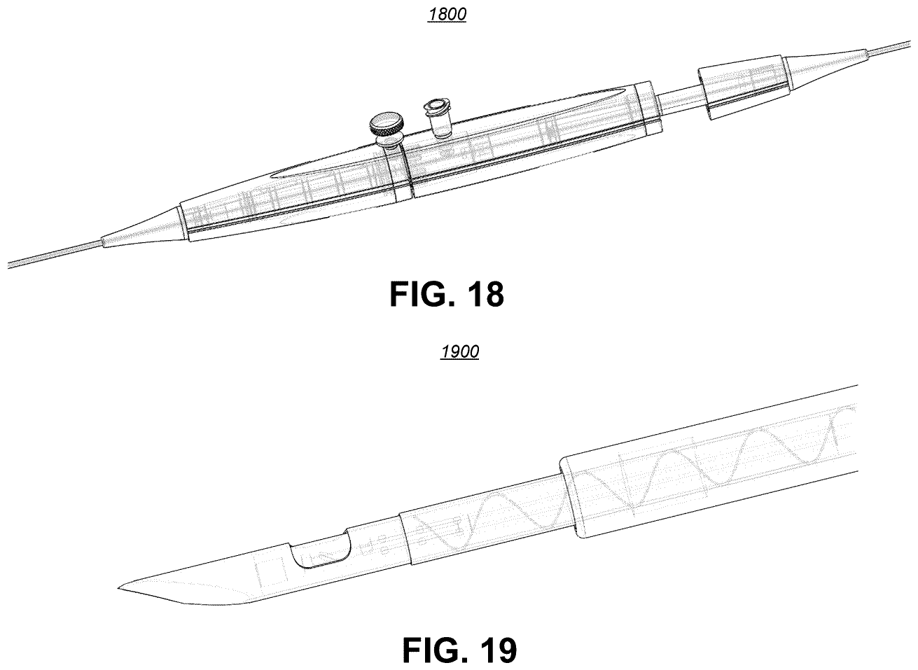

In an image-guided biopsy mode, the biopsy device 100 is configured to image tissue at a target area of the human body, such as by puncturing a target nodule with the needle 300 and imaging within. FIG. 18 illustrates the segmented handle 700 of the biopsy device 100 in the image-guided biopsy operating mode. FIG. 19 illustrates the distal end of the outer sheath 600, including the needle 300 and imaging stylet 400 projecting outward from the outer sheath 600. To select the image-guided biopsy operating mode (provided that the biopsy device 100 is initially in the imaging operating mode), the user translationally moves the second handle piece 720 toward the distal end of the biopsy device 100 such that the second handle piece 720 abuts or nearly abuts the first handle piece 710, causing the needle 300 to project outward from the outer sheath 600. The third handle piece 730 remains stationary at its position in the imaging operating mode, causing the needle 300 to slide over the imaging stylet 400 and aligning a lens of the imaging stylet 400 with the imaging window in the side of the needle 300. The translational movement of the second handle piece 720 may be a single rapid movement to drive the needle 300 into the target tissue area of the human body and puncture the tissue. Once the tissue is punctured, the imaging stylet 400 can image the target tissue area from within.

In some cases, the user may first unlock (e.g., unscrew) the locking mechanism 740 of the first handle piece 710 to allow for translational movement of the second handle piece 720. The user may also install a clip (e.g., a c-clip) at a desired demarcation 750 on the second handle piece 720 to prevent the needle 300 from projecting fully from the outer sheath 600, limiting its projection depth into the target tissue area. For example, the target tissue area may be a nodule 2 centimeters in diameter, so the user can install the c-clip at a 1-centimeter demarcation on the second handle piece 720 to prevent the needle 300 from puncturing deeper than 1 centimeter into the nodule.

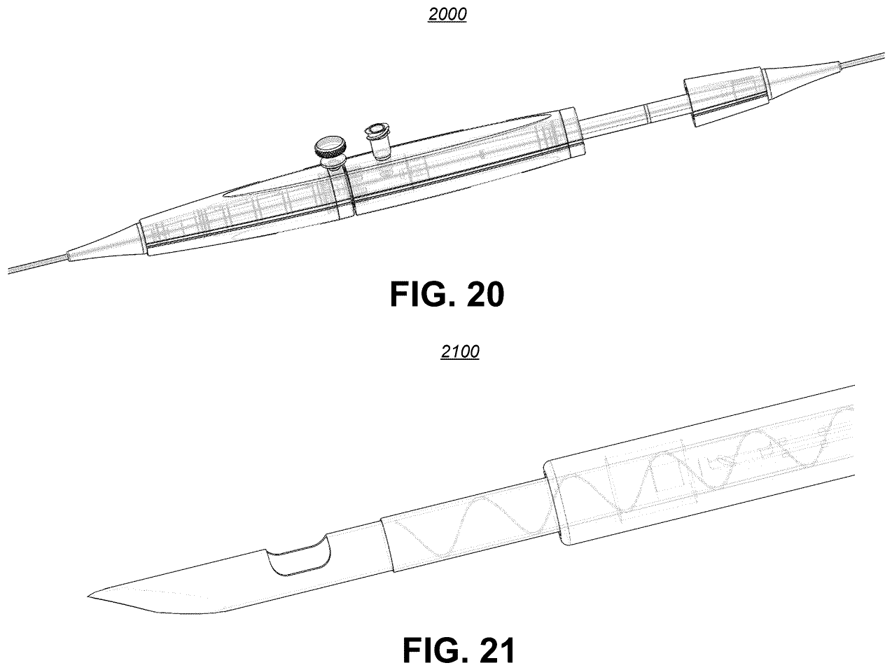

In a biopsy operating mode, the biopsy device 100 is configured to perform a biopsy on the target tissue area. FIG. 20 illustrates the segmented handle 700 of the biopsy device 100 in the biopsy operating mode. FIG. 21 illustrates the distal end of the outer sheath 600, including the needle 300 projecting outward from the outer sheath 600. The biopsy operating mode may be used to acquire tissue samples from the target tissue area. To select the biopsy operating mode (provided that the biopsy device 100 is initially in the image-guided biopsy operating mode), the user translationally moves the third handle piece 730 to a maximum position of its range of movement toward the proximal end of the biopsy device 100, causing the imaging stylet 400 to vacate the needle tip and be positioned within the outer sheath. This leaves the needle tip projecting from the outer sheath 600, empty and ready to accept tissue samples. The user can connect an aspiration syringe with a pre-loaded negative pressure to the Luer connector 760 of the second handle piece 720 to create a vacuum within the needle 300. The user translationally moves the second handle piece 720 in a reciprocating motion to agitate the needle 300 and collect a tissue sample within the needle tip.

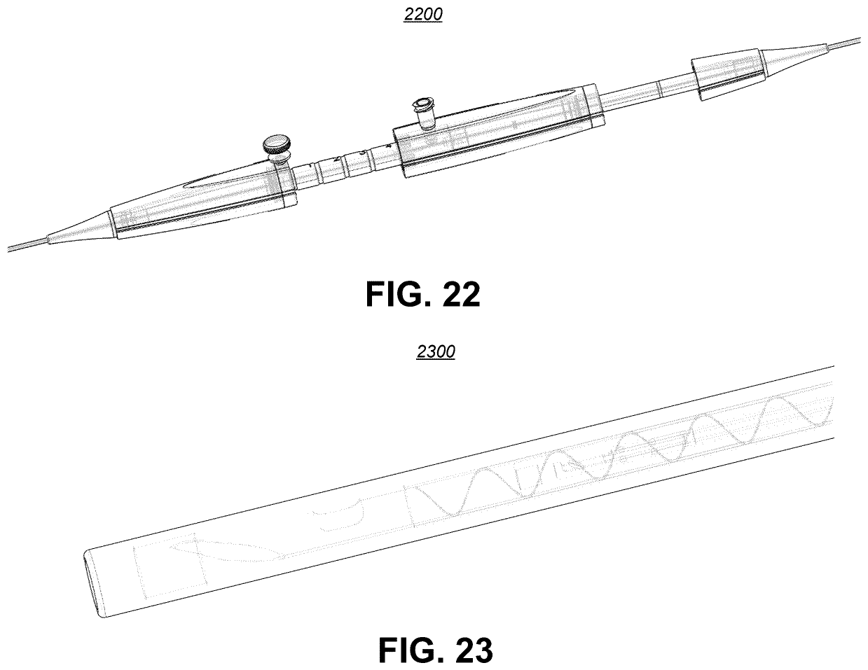

In a retrieval operating mode, the user prepares the biopsy device for retrieval of the tissue sample and removal of the device 100 from the bronchoscope working channel. FIG. 22 illustrates the segmented handle 700 of the biopsy device 100 in the retrieval operating mode. FIG. 23 illustrates the distal end of the outer sheath 600, including the needle 300 and the imaging stylet 400 located within the outer sheath 600. To select the retrieval operating mode (provided that the biopsy device 100 is initially in the biopsy operating mode), the user translationally moves the second handle piece 720 toward the proximal end of the biopsy device 100, causing the needle 300 to retract within the outer sheath 600.

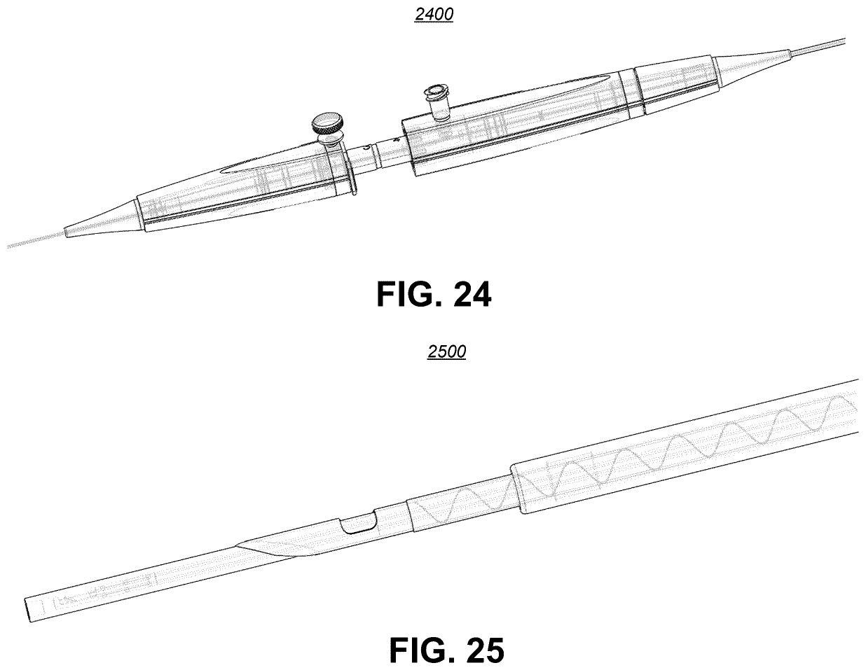

In an ejection operating mode, the biopsy device 100 ejects the tissue biopsy sample from the needle 300. FIG. 24 illustrates the segmented handle 700 of the biopsy device 100 in the ejection operating mode. FIG. 25 illustrates the distal end of the outer sheath 600, including the needle 300 projecting outward from the outer sheath 600 and the imaging stylet 400 projecting outward from the needle 300. The ejection operating mode is used to remove the tissue samples from the needle 300 once the biopsy device 100 has been removed from the human body. To select the ejection operating mode (provided that the biopsy device 100 is initially in the retrieval operating mode), the user translationally moves the third handle piece 730 toward the distal end of the biopsy device 100 such that the third handle piece 730 abuts the second handle piece 720, causing the imaging stylet 400 to project outward from the needle 300, and translationally moves the second handle piece 720 and the third handle piece 730 toward the distal end of the biopsy device 100, causing the needle 300 and imaging stylet 400 to project outward from the outer sheath 600. The user may also disconnect the negative pressure syringe from the Luer connector of the second handle piece 720 when the tissue sample is ready to eject from the needle 300. The user may also flush the tissue out by connecting a saline syringe to the Luer connector and flushing saline through the needle.

### Example Communication Modalities

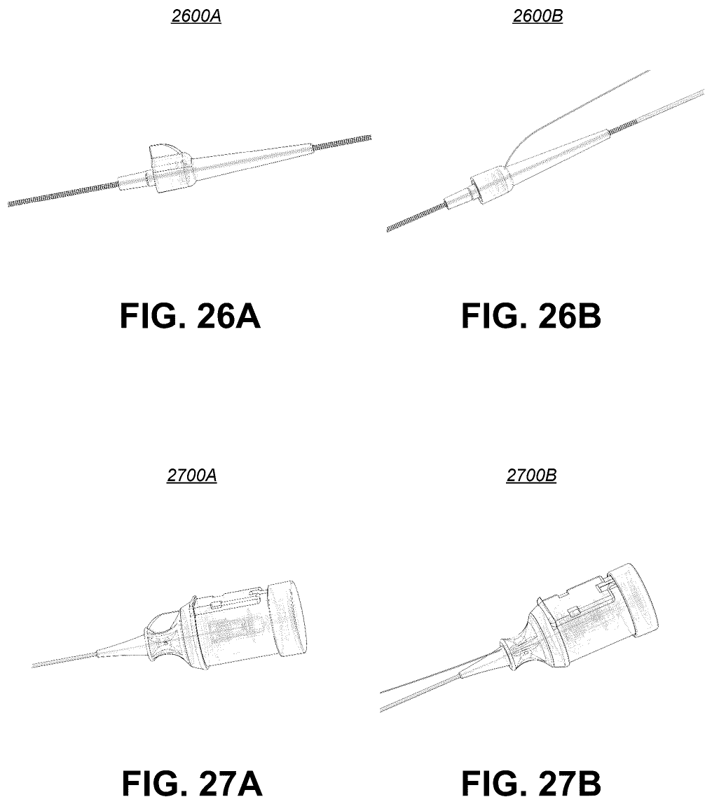

FIG. 26A illustrates a strain relief module 2600A of a biopsy device in accordance with a wireless communications embodiment. FIG. 26B illustrates a strain relief module 2600B of the biopsy device in accordance with a wired communications embodiment. The strain relief modules 2600 may be used to dock the biopsy device to an entry port of a bronchoscope to provide more stability during imaging and biopsy. In various embodiments, the strain relief modules 2600 includes an encoder (e.g., an electromagnetic encoder, a rotary encoder, or an optical encoder) configured to track movement of the biopsy device, such as forward and backward movement of the biopsy device 100 relative to the bronchoscope. For example, the outer sheath 600 may include a plurality of demarcations configured to be read by an optical encoder as the biopsy device is being fed into the bronchoscope. Based on the number of demarcations which are detected by the optical encoder, the encoder can generate movement data indicating a current depth of the biopsy device.

FIG. 27A illustrates a driving unit 2700A in accordance with the wireless communications embodiment. FIG. 27B illustrates a driving unit 2700B in accordance with the wired communications embodiment. The driving units 2700 are similar to the driving unit 800 of FIG. 8 and further include a receiver encoder configured to receive movement data of the biopsy device 100 from one of the strain relief modules 2600. An imaging console communicatively connected to one of the driving units 2700 receives the movement data, and may combine the movement data with imaging data generated by the imaging stylet 400 to assist in navigating an airway or other luminal organ of the human body.

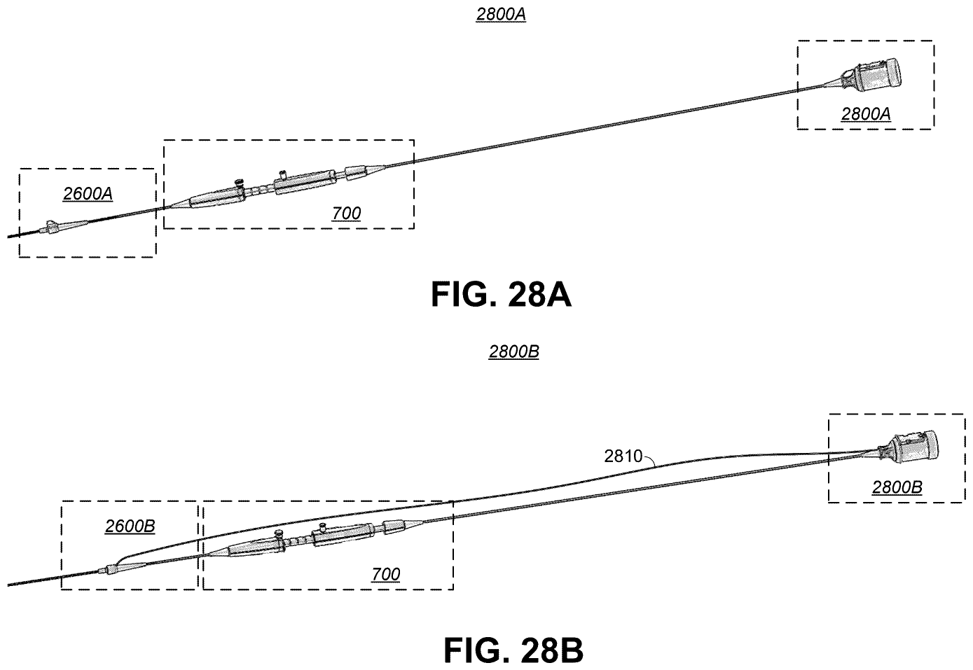

FIG. 28A illustrates a biopsy device 2800A, including the strain relief module 2600A and the driving unit 2700A, in accordance with the wireless communications embodiment. FIG. 28B illustrates a biopsy device 2800B, including the strain relief module 2600B and the driving unit 2700B, in accordance with the wired communications embodiment. In both embodiments, the strain relief module 2600A or 2600B is configured to transmit movement data to a receiver encoder of a corresponding one of the driving units 2700. In the wireless communications embodiment, the movement data is wirelessly transmitted by an antenna of the strain relief module 2600A to a wireless receiver encoder of the driving unit 2700A. In the wired communications embodiment, the movement data is transmitted over a wire 2810 which physically connects the strain relief module 2600B and the driving unit 2700B. In another embodiment, the movement data can be transmitted from a strain relief transmitter directly to the imaging console.

### Example Computing Device Architecture

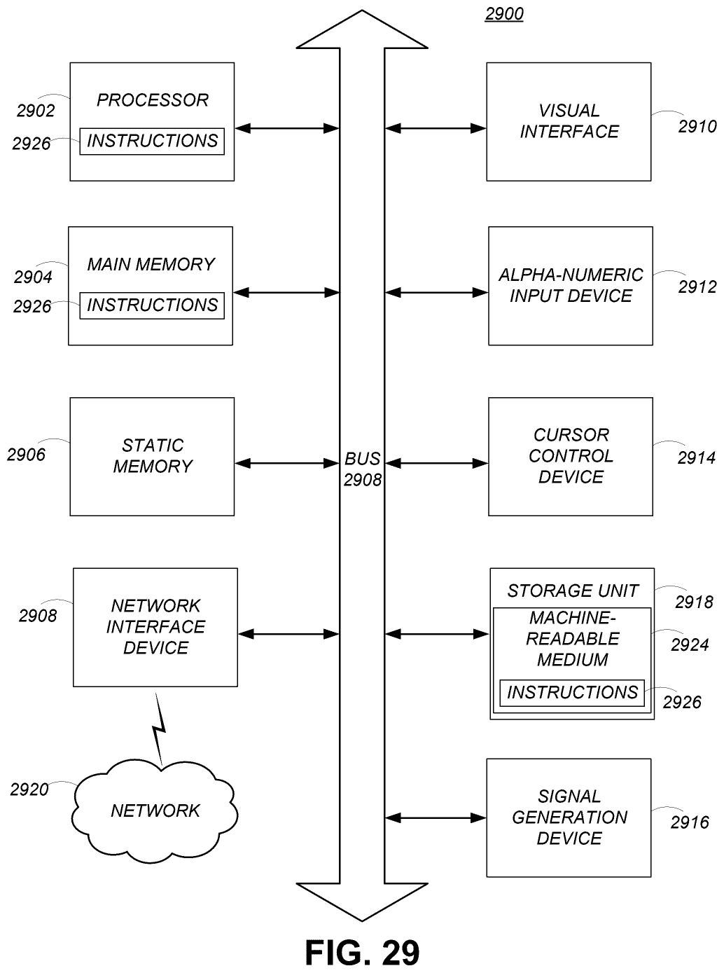

FIG. 29 illustrates an example machine of a computer system 2900 within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 2900 includes a processing device 2902, a main memory 2904 (e.g., read-only memory (ROM), flash memory, dynamic random access memory (DRAM) such as synchronous DRAM (SDRAM), a static memory 2906 (e.g., flash memory, static random access memory (SRAM), etc.), and a data storage device 2918, which communicate with each other via a bus 2930.

Processing device 2902 represents one or more processors such as a microprocessor, a central processing unit, or the like. More particularly, the processing device may be complex instruction set computing (CISC) microprocessor, reduced instruction set computing (RISC) microprocessor, very long instruction word (VLIW) microprocessor, or a processor implementing other instruction sets, or processors implementing a combination of instruction sets. Processing device 2902 may also be one or more special-purpose processing devices such as an application specific integrated circuit (ASIC), a field programmable gate array (FPGA), a digital signal processor (DSP), network processor, or the like. The processing device 2902 may be configured to execute instructions 2926 for performing the operations and steps described herein.

The computer system 2900 may further include a network interface device 2908 to communicate over the network 2920. The computer system 2900 also may include a video display unit 2910 (e.g., a liquid crystal display (LCD) or a cathode ray tube (CRT)), an alphanumeric input device 2912 (e.g., a keyboard), a cursor control device 2914 (e.g., a mouse), a graphics processing unit 2922, a signal generation device 2916 (e.g., a speaker), graphics processing unit 2922, video processing unit 2928, and audio processing unit 2932.

The data storage device 2918 may include a machine-readable storage medium 2924 (also known as a non-transitory computer-readable medium) on which is stored one or more sets of instructions 2926 or software embodying any one or more of the methodologies or functions described herein. The instructions 2926 may also reside, completely or at least partially, within the main memory 2904 and/or within the processing device 2902 during execution thereof by the computer system 2900, the main memory 2904 and the processing device 2902 also constituting machine-readable storage media.

In some implementations, the instructions 2926 include instructions to implement functionality corresponding to the present disclosure. While the machine-readable storage medium 2924 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine and the processing device 2902 to perform any one or more of the methodologies of the present disclosure. The term “machine-readable storage medium” shall accordingly be taken to include, but not be limited to, solid-state memories, optical media, and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm may be a sequence of operations leading to a desired result. The operations are those requiring physical manipulations of physical quantities. Such quantities may take the form of electrical or magnetic signals capable of being stored, combined, compared, and otherwise manipulated. Such signals may be referred to as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the present disclosure, it is appreciated that throughout the description, certain terms refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may include a computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various other systems may be used with programs in accordance with the teachings herein, or it may prove convenient to construct a more specialized apparatus to perform the method. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. Where the disclosure refers to some elements in the singular tense, more than one element can be depicted in the figures and like elements are labeled with like numerals. The disclosure and drawings are, accordingly, to be regarded in an illustrative sense rather than a restrictive sense.

What is claimed is:

1. A device to facilitate imaging within a human body, comprising:
a handle positioned toward a proximal end of the device, the handle comprising a first handle piece, a second handle piece, and a third handle piece, the handle pieces being adjustable between a plurality of states to control a plurality of operating modes of the device based on translational and rotational motion of the first handle piece, the second handle piece, and the third handle piece;
a sheath within the device, the sheath operably connected to a first handle piece such that the first handle piece controls the translational motion of the sheath within the device;
a needle within the sheath, the needle comprising a needle tip of the needle positioned toward a distal end of the device, and the needle being operably connected to the second handle piece such that the second handle piece controls translational and rotational motion of the needle within the sheath; and
an imaging stylet positioned within the needle, the imaging stylet comprising an imaging assembly and an optically transmissive housing for imaging within the human body, and the imaging stylet being operably connected to the third handle piece such that the third handle piece controls translational and rotational motion of the imaging stylet within the sheath.

2. The device of claim 1, further comprising a fourth handle piece configured for rotational motion and operably connected to a steerable articulating segment of the sheath, the steerable articulating segment configured to guide the needle to a target within the human body.

3. The device of claim 1, further comprising a driving unit positioned at a proximal end of the device, the driving unit including:
an optically transmissive fiber for rotating the imaging assembly perform 360-degree imaging; and
an optical connector having a textured pattern formed in a wall of the optical connector, the pattern encoding a unique identifier of the device.

4. The device of claim 1 wherein, in a home operating mode, the third handle piece is translationally moved apart from the second handle piece toward the proximal end of the device, causing the imaging stylet to be positioned within the needle tip, and the second handle piece is translationally moved apart from the first handle piece toward the proximal end of the device, causing the needle tip to be positioned within the sheath.

5. The device of claim 1 wherein, in an imaging operating mode, the third handle piece is translationally moved toward the distal end of the device such that the third handle piece abuts the second handle piece, causing the imaging stylet to project outward from the needle and the sheath for imaging.

6. The device of claim 1 wherein, in an image-guided biopsy operating mode, the second handle piece is translationally moved toward the distal end of the device such that the second handle piece abuts the first handle piece, causing the needle to project outward from the sheath, and the third handle piece remains stationary toward the proximal end of the device, aligning a lens of the imaging assembly with a discontinuity in the needle tip.

7. The device of claim 1 wherein, in a biopsy operating mode, the third handle piece is moved to a maximum position of its range of translational movement toward the proximal end of the device, causing the imaging stylet to vacate the needle tip and be positioned within the sheath, and the second handle piece is translationally moved in a reciprocating motion to agitate the needle and collect a tissue sample within the needle tip.

8. The device of claim 1 wherein, in an ejection operating mode, the third handle piece is translationally moved toward the distal end of the device such that the third handle piece abuts the second handle piece, causing the imaging stylet to eject a tissue sample from the needle tip.

9. The device of claim 1, wherein the imaging stylet is capable of three degrees of translational motion and two degrees of rotational motion.

10. A method comprising:
adjusting a handle comprising a first handle piece, a second handle piece, and a third handle piece between a plurality of states to control a plurality of operating modes of a biopsy device based on translational and rotational motion of the first handle piece, the second handle piece, and the third handle piece;
wherein a sheath within the device is operably connected to the first handle piece such that the first handle piece controls translational motion of the sheath within the device;
wherein a needle within the sheath is operably connected to the second handle piece such that the second handle piece controls translational and rotational motion of the needle within the sheath, the needle including a needle tip; and
wherein an imaging stylet within the needle is operably connected to the third handle piece such that the third handle piece controls translational and rotational motion of the imaging stylet, the imaging stylet comprising an imaging assembly and an optically transmissive housing for imaging within a human body.

11. The method of claim 10, further comprising adjusting a fourth handle piece of the device configured for rotational motion and operably connected to a steerable articulating segment of the sheath, the steerable articulating segment configured to guide the needle to a target within the human body.

12. The method of claim 10, further comprising rotating, by an optically transmissive fiber, the imaging assembly to perform 360-degree optical coherence tomography (OCT) imaging.

13. The method of claim 10, further comprising selecting a home operating mode for the device by translationally moving the third handle piece apart from the second handle piece toward a proximal end of the device, causing the imaging stylet to be positioned within the needle tip, and translationally moving the second handle piece apart from the first handle piece toward a proximal end of the device, causing the needle tip to be positioned within the sheath.

14. The method of claim 10, further comprising selecting an imaging operating mode for the device by translationally moving the third handle piece toward a distal end of the device such that the third handle piece abuts the second handle piece, causing the imaging stylet to project outward from the needle and the sheath for imaging.

15. The method of claim 10, further comprising selecting an image-guided biopsy operating mode for the device by translationally moving the second handle piece toward a distal end of the device such that the second handle piece abuts the first handle piece, causing the needle to project outward from the sheath, and maintaining a position of the third handle piece toward a proximal end of the device, aligning a lens of the imaging assembly with a discontinuity in the needle tip.

16. The method of claim 10, further comprising selecting a biopsy operating mode for the device by translationally moving the third handle piece to a maximum position of its range of translational movement toward a proximal end of the device, causing the imaging stylet to vacate the needle tip and be positioned within the sheath, and translationally moving the second handle piece in a reciprocating motion to agitate the needle and collect a tissue sample within the needle tip.

17. The method of claim 10, further comprising selecting an ejection operating mode for the device by translationally moving the third handle piece toward a distal end of the device such that the third handle piece abuts the second handle piece, causing the imaging stylet to eject a tissue sample from the needle tip.

18. The method of claim 10, further comprising moving the imaging stylet along three axes of translational motion and two axes of rotational motion.

19. The method of claim 10, further comprising adjusting an angle of curvature of the sheath by translational or rotational movement of a control arm of a steering assembly of the device.

20. The method of claim 10, further comprising tracking movement of the device within the human body by accessing movement data generated by an encoder and imaging data generated by the imaging stylet.
